(19) United States
(12) Patent Application Publication
(10) Pub. No.: US 2024/0390290 A1
(43) Pub. Date: Nov. 28, 2024

(54) ISO-OSMOTIC AND NEAR ISO-OSMOTIC OXYGEN THERAPEUTIC FORMULATIONS AND METHODS THEREOF

(71) Applicant: NuvOx Pharma LLC, Tucson, AZ (US)

(72) Inventors: Evan C. Unger, Tucson, AZ (US); Edmund R. Marinelli, Tucson, AZ (US)

(21) Appl. No.: 18/648,420

(22) Filed: Apr. 28, 2024

Related U.S. Application Data
(63) Continuation of application No. 16/479,589, filed on Jul. 20, 2019, now abandoned.
(60) Provisional application No. 62/450,050, filed on Jan. 24, 2017.

(51) Int. Cl. A61K 31/02; A61K 9/107; A61K 47/10; A61K 47/26

(52) U.S. Cl. CPC A61K 31/02; A61K 9/1075; A61K 47/10; A61K 47/26

(57) ABSTRACT

The invention provides novel iso-osmotic or near iso-osmotic formulations of fluorocarbon emulsions that are useful as oxygen therapeutics, for example, for treating sickle cell disease and related diseases and conditions, as well as methods of preparation and use thereof.

### PRIORITY CLAIMS AND RELATED PATENT APPLICATIONS

This application claims the benefit of priority to U.S. Provisional Application Ser. No. 62/450,050, filed on Jan. 24, 2017, the entire content of which is incorporated herein by reference.

### TECHNICAL FIELDS OF THE INVENTION

This invention relates to pharmaceutical compositions and methods of their preparation and therapeutic use. More particularly, the invention relates to iso-osmotic or near iso-osmotic formulations of fluorocarbon emulsions that are useful as oxygen therapeutics, for example, for treating sickle cell disease and related diseases and conditions, as well as methods of preparation and use thereof.

### BACKGROUND OF THE INVENTION

Plasma osmolality measures the body's electrolyte-water balance. The normal human reference range of osmolality in plasma is about 285-295 milliosmoles per kilogram. Certain diseases can be exacerbated by solutions that are not iso-osmotic or isotonic (e.g., by solutions that are hypotonic or hypertonic). In oxygen therapeutics it is generally preferable to include viscogens to stabilize the formulation, e.g., to stabilize an emulsion of a liquid fluorocarbon. Sucrose, for example, can be used as a viscogen. In the prior art, formulations with viscogens had osmolality higher than that of the plasma. For example, 2% wt/vol perfluoropentane (DDFP) emulsion with 0.3 wt/vol % PEG-Telomer-B in phosphate buffered 30% aqueous sucrose solution) has an osmolality of approximately 1,000 milliosmoles/kg, which is more than 3 times hypertonic to plasma.

Administration of hypertonic solutions can cause red blood cells to crenate and predisposes to certain conditions. In sickle cell disease administration of hypertonic solutions increases the risk of red blood cell sickling.

Thus, an urgent need remains for safe and reliable oxygen therapeutics, in particular, that are formulated to address these concerns.

### SUMMARY OF THE INVENTION

The present invention is based in part on the discovery of novel iso-osmotic or near iso-osmotic solutions of oxygen therapeutics.

In one aspect, the invention generally relates to an oxygen therapeutic composition. The oxygen therapeutic composition includes a fluorocarbon compound comprising about 4 to about 8 carbon atoms, a surfactant, a viscogen and water. The oxygen therapeutic composition is an emulsion of particles comprising the fluorocarbon and is characterized by an osmolality in the range from about 200 milliosmoles/kg to about 500 milliosmoles/kg.

In another aspect, the invention generally relates to a method for treating sickle cell disease, or a related disease or condition, comprising administering to a subject in need thereof an oxygen therapeutic composition. The oxygen therapeutic composition includes a fluorocarbon compound comprising about 4 to about 8 carbon atoms, a surfactant, and saline in a concentration from about 0.6 to about 1.5%. The oxygen therapeutic composition is an emulsion of particles comprising the fluorocarbon and is characterized by an osmolality in the range from about 200 milliosmoles/kg to about 500 milliosmoles/kg.

In yet another aspect, the invention generally relates to a method for treating a disease or condition comprising administering to a subject in need thereof the oxygen therapeutic composition disclosed herein. In certain embodiments, the disease or condition is sickle cell disease or a related disease and condition.

In yet another aspect, the invention generally relates to a method for treating sickle cell disease, or a disease or condition. The method includes administering to a subject in need thereof an oxygen therapeutic composition. The oxygen therapeutic composition includes a fluorocarbon compound comprising about 4 to about 8 carbon atoms, a surfactant, a viscogen and water. The oxygen therapeutic composition in the range from about 200 milliosmoles/kg to about 500 milliosmoles/kg.

In yet another aspect, the invention generally relates to a method for treating sickle cell disease, or a related disease or condition, comprising administering to a subject in need thereof an oxygen therapeutic composition. The oxygen therapeutic composition includes a fluorocarbon compound comprising about 4 to about 8 carbon atoms, a surfactant, and saline in a concentration from about 0.6% to about 1.5%. The oxygen therapeutic composition is an emulsion of particles comprising the fluorocarbon and is characterized by an osmolality in the range from about 200 milliosmoles/kg to about 500 milliosmoles/kg.

### DETAILED DESCRIPTION OF THE INVENTION

The invention provides a novel iso-osmotic or near iso-osmotic oxygen therapeutic composition comprising a fluorocarbon emulsion with an osmolality in the range from about 200 milliosmoles/kg to about 500 milliosmoles/kg.

Osmolality and osmolarity are units of measurement for body's electrolyte-water balance. There are slight differences in measured values for osmolality and osmolarity but the terms are used interchangeably herein for the purpose of this disclosure.

Osmolality is the number of osmoles of solute in a kilogram of solvent, while osmolarity is the number of osmoles of solute in a liter of solution. Osmolality measures the number of particles in the unit weight of a solvent, and is independent of the shape, size or weight of the particles. Osmolarity is the concentration of an osmotic solution. The volume of a solution will change with the addition of solutes, and also with any change in temperature or pressure. The difference between the calculated osmolarity and measured osmolality is known as the osmolar gap.

In one aspect, the invention generally relates to an oxygen therapeutic composition. The oxygen therapeutic composition includes: a fluorocarbon compound comprising about 4 to about 8 carbon atoms, a surfactant, a viscogen and water. The oxygen therapeutic composition is an emulsion of particles comprising the fluorocarbon and is characterized by an osmolality in the range from about 200 milliosmoles/kg to about 500 milliosmoles/kg.

In certain embodiments, the fluorocarbon is a perfluorocarbon compound.

In certain embodiments, the perfluorocarbon compound is selected from the group consisting of perfluorobutane, perfluoropentane and perfluorohexane.

In certain embodiments, the perfluorocarbon compound is perfluoropentane.

Preferably, the osmolality of the oxygen therapeutic formulation is in the range from about 200 milliosmoles/kg to about 500 milliosmoles/kg. More preferably, the osmolality of the formulation is in the range from about 240 milliosmoles/kg to about 340 milliosmoles/kg, e.g., from about 265 milliosmoles/kg to about 315 milliosmoles/kg. Even more preferably, the osmolality of the formulation is in the range from about 275 milliosmoles/kg to about 305 milliosmoles/kg. Most preferably, the osmolality of the formulation is in the range from about 285 milliosmoles/kg to about 295 milliosmoles/kg.

Preferably, the particles have particle sizes: intensity weighted mean diameter (IWMD) in the range from about 100 nm to about 600 nm (e.g., from about 100 nm to about 500 nm, from about 100 nm to about 400 nm, from about 100 nm to about 300 nm, from about 200 nm to about 600 nm, from about 300 nm to about 600 nm, from about 400 nm to about 600 nm, from about 200 nm to about 400 nm, from about 300 nm to about 500 nm).

In certain embodiments, the viscogen is a polyether, for example, a polyethylene glycol (PEG).

Preferably, the polyethylene glycol has an average molecular weight Mₙ less than 1,000 (e.g., between about 200 and about 800, between about 200 and about 500, between about 200 and about 400, between about 300 and about 400).

In certain embodiments, the polyether (e.g., polyethylene glycol) is present in the composition at a concentration from about 1 w/v % to about 30 w/v %, preferably at a concentration from about 2 w/v % to about 15 w/v %, more preferably at a concentration from about 5 w/v % to about 15 w/v %.

In certain embodiments, the viscogen is a polyol (e.g., maltitol, erythritol, lactitol, xylitol, sorbitol, mannitol, isomalt and polyglycitol) or a combination of polyols.

In certain embodiments, the polyol (e.g., maltitol) is present in the composition at a concentration from about 1 w/v % to about 30 w/v %, preferably at a concentration from about 2 w/v % to about 15 w/v %, more preferably at a concentration from about 5 w/v % to about 15 w/v %.

In certain embodiments, the viscogen comprises a combination of a polyether compound and a polyol compound.

In certain embodiments, the polyether (e.g., polyethylene glycol) and the polyol (e.g., maltitol) are present in the composition at a total concentration from about 1 w/v % to about 30 w/v %, preferably at a concentration from about 2 w/v % to about 15 w/v %, more preferably at a concentration from about 5 w/v % to about 15 w/v %.

The polyols, singly or in combination, (e.g., maltitol, erythritol, lactitol, xylitol, sorbitol, mannitol, isomalt and polyglycitol) can act as both the osmolyte and as a viscogen. Maltitol has an advantage over sucrose (or certain other disaccharides), for example, in that maltitol has about half the glycemic index and insulinemic index of sucrose at the same concentration. This is a clear advantage when administering a therapeutic composition to diabetic patients, especially in multi-dose and/or extended use scenarios.

In certain embodiments, the oxygen therapeutic composition is free of disaccharides (i.e., does not comprise disaccharides).

In certain embodiments, the oxygen therapeutic composition is free of sucrose (i.e., does not contain sucrose).

In another aspect, the invention generally relates to an oxygen therapeutic composition. The oxygen therapeutic composition includes a fluorocarbon compound comprising about 4 to about 8 carbon atoms, a surfactant, and saline in a concentration from about 0.6% to about 1.5%. The oxygen therapeutic composition is an emulsion of particles comprising the fluorocarbon, and is characterized by an osmolality in the range from about 200 milliosmoles/kg to about 500 milliosmoles/kg.

In certain embodiments, the fluorocarbon is a perfluorocarbon compound (e.g., perfluorobutane, perfluoropentane or perfluorohexane).

Saline is preferably present at a concentration from about 0.6 w/v % to about 1.5 w/v %, more preferably at a concentration from about 0.7 w/v % to about 1.2 w/v %, even more preferably at a concentration from about 0.8 w/v % to about 1.0 w/v %.

Preferably, the osmolality of the oxygen therapeutic formulation is in the range from about 200 milliosmoles/kg to about 500 milliosmoles/kg. More preferably, the osmolality of the formulation is in the range from about 240 milliosmoles/kg to about 340 milliosmoles/kg, e.g., from about 265 milliosmoles/kg to about 315 milliosmoles/kg. Even more preferably, the osmolality of the formulation is in the range from about 275 milliosmoles/kg to about 305 milliosmoles/kg. Most preferably, the osmolality of the formulation is in the range from about 285 milliosmoles/kg to about 295 milliosmoles/kg.

Preferably, the particles have particle sizes: IWMD in the range from about 100 nm to about 600 nm (e.g., from about 100 nm to about 500 nm, from about 100 nm to about 400 nm, from about 100 nm to about 300 nm, from about 200 nm to about 600 nm, from about 300 nm to about 600 nm, from about 400 nm to about 600 nm, from about 200 nm to about 400 nm, from about 300 nm to about 500 nm).

In certain embodiments, the viscogen is sucrose and is present at a concentration less than about 15 w/v % (e.g., about 2 w/v % to about 10 w/v %, about 5 w/v % to about 10 w/v %).

In certain embodiments, the oxygen therapeutic composition comprises PEG-Telomer B (PTB) as a surfactant.

In certain embodiments, the oxygen therapeutic composition is free of a viscogen.

Formulations of the invention using only saline as the osmolyte and using no viscogen can be advantageous when a patient is allergic to a viscogen or is to avoid risk of allergic reactions to any given viscogen. It was unexpectedly found that the particle sizes can be kept stable even in the absence of a viscogen.

In certain embodiments the oxygen therapeutic composition is free of, or contains only very small amounts of ionic components-an example would be 5 mM sodium phosphate buffer with no other ionic species such as saline. In this case the concentration of the viscogen can be adjusted to provide both the desired viscosity and osmolality of the composition.

In certain embodiments, the oxygen therapeutic composition is stabilized by one or more surfactants. For example, surfactants may be one or more fluorosurfactants such as PEG-Telomer-B, CAPSTONE, diacylglycerophospholipids, cholesterol, and/or other surfactants known in the art. In certain embodiments, the surfactant(s) utilized comprise one or more fluorosurfactants and one or more phospholipids. In certain embodiments, the surfactant(s) is incorporated into the nanoemulsion in amounts ranging from about 0.1% weight volume to about 10% weight volume. In certain embodiments, the surfactant(s) is incorporated into the nanoemulsion in amounts ranging from about 0.1% w/vol to about 5% w/vol. In certain embodiments, the surfactant(s) is incorporated into the nanoemulsion in amounts ranging from about 0.2% w/vol to about 2% w/vol.

As an example, Capstone FS-3100 surfactant is a perfluorocarbon-oligoethoxyalcohol surfactant produced by Dupont Co. The structure consists of at least 90% of F6 that is a straight chain perfluorohexyl moiety CF₃CF₂CF₂CF₂CF₂CF₂—. Small amounts of perfluorobutyl (F4) and perfluoroethyl (F2) congeners may be present in the product. The oligoethyleneoxyalcohol function is a distribution of oligoethylene alcohol functions (CH₂CH₂O)ₙ where n=2,3,4,5,6,7, 8, 9, 10, 11, 12, 13 and 14 are present with the combined amounts of materials where n>8 constituting less than 5%. (DuPont™ CAPSTONE® Repellents and Surfactants—An Overview; Cadet, et al. US Pub. No. 2014/0177053 A1; Cadet, et. al. U.S. Pat. No. 9,645,285 B2.)

PEG-Telomer-B is a custom purified perfluorocarbon-oligoethyleneoxyalcohol surfactant that is obtained starting with Dupont Zonyl FSO-100 or Dupont Zonyl FSO. The purified product contains a mixture of F4, F6, F8, F10, F12, F14 and F16 compounds in the approximate ranges of relative amounts. F4≤30.3%, F6 53-69%, F8 24-36%, F10 5-11%, F12, F14 and F16 combined ≤1.6%. Very small amounts of perfluoroethyl (F2) congener may be present in the product. Here, F refers to the number of perfluorinated carbons in the perfluorocarbon moiety present. The oligoethyleneoxyalcohol function is a distribution of oligoethylene alcohol functions (CH₂CH₂₀)ₙ where n=2, 3, 4, 5, 6, 7, 8, 9, 10, 11, 12, 13, 14, 15, or 16 are present. Very small amounts of n>16 may be present in the product.

In another aspect, the invention generally relates to a method for treating a disease or condition comprising administering to a subject in need thereof the oxygen therapeutic composition disclosed herein. In certain embodiments, the disease or condition is sickle cell disease, or a related disease and condition.

In yet another aspect, the invention generally relates to a method for treating sickle cell disease, or a related disease or condition. The method includes administering to a subject in need thereof an oxygen therapeutic composition. The oxygen therapeutic composition includes a fluorocarbon compound comprising about 4 to about 8 carbon atoms, a surfactant and a viscogen. The oxygen therapeutic composition is characterized by an osmolality in the range from about 200 milliosmoles/kg to about 500 milliosmoles/kg.

In yet another aspect the invention generally relates to a method for treatment of a condition requiring the oxygen therapeutic wherein supplementation of electrolytes (which may include saline) to the patient is being conducted concurrently, thus precluding the oxygen therapeutic from adding to the electrolyte load being administered to the patient, hence an iso-osmotic formulation substantially devoid of electrolytes is required.

The fluorocarbon is preferably completely fluorinated. In certain embodiments, the fluorocarbon is selected from the group consisting of perfluorobutane, perfluoropentane and perfluorohexane. Preferably, the fluorocarbon is perfluoropentane.

Preferably, the osmolality of the oxygen therapeutic formulation is in the range from about 200 milliosmoles/kg to about 500 milliosmoles/kg. More preferably, the osmolality of the formulation is in the range from about 240 milliosmoles/kg to about 340 milliosmoles/kg, e.g., from about 265 milliosmoles/kg to about 315 milliosmoles/kg. Even more preferably, the osmolality of the formulation is in the range from about 275 milliosmoles/kg to about 305 milliosmoles/kg. Most preferably, the osmolality of the formulation is in the range from about 285 milliosmoles/kg to about 295 milliosmoles/kg.

Preferably, the particles have a particle size: IWMD in the range from about 100 nm to about 600 nm (e.g., from about 100 nm to about 500 nm, from about 100 nm to about 400 nm, from about 100 nm to about 300 nm, from about 200 nm to about 600 nm, from about 300 nm to about 600 nm, from about 400 nm to about 600 nm, from about 200 nm to about 400 nm, from about 300 nm to about 500 nm).

In certain embodiments, the viscogen is a polyethylene glycol having an average molecular weight Mₙ less than 1,000 (e.g., between about 200 and about 800, between about 200 and about 500, between about 200 and about 400, between about 300 and about 400).

In certain embodiments, the polyethylene glycol is present in the composition at a concentration from about 1 w/v % to about 30 w/v %, preferably at a concentration from about 2 w/v % to about 15 w/v %, more preferably at a concentration from about 5 w/v % to about 15 w/v %.

In certain embodiments, the viscogen is a polyol (e.g., maltitol, erythritol, lactitol, xylitol, sorbitol, mannitol, isomalt and polyglycitol) or any combination thereof.

In certain embodiments, the polyol is present in the composition at a concentration from about 1 w/v % to about 30 w/v %, preferably at a concentration from about 2 w/v % to about 15 w/v %, more preferably at a concentration from about 5 w/v % to about 15 w/v %.

In certain embodiments, the viscogen comprises both a polyether and a polyol.

In certain embodiments, the oxygen therapeutic composition is free of disaccharides (i.e., does not comprise disaccharides).

In certain embodiments, the oxygen therapeutic composition is free of sucrose (i.e., does not comprise sucrose).

In certain embodiments, the subject is a diabetic or otherwise needs to avoid administration of sucrose.

In certain embodiments, the viscogen is sucrose and is present at a concentration less than about 15 w/v % (e.g., about 2 w/v % to about 10 w/v %, about 5 w/v % to about 10 w/v %).

In certain embodiments, the oxygen therapeutic composition comprises PEG-Telomer B (PTB) as a surfactant.

In yet another aspect, the invention generally relates to a method for treating sickle cell disease, or a related disease or condition, comprising administering to a subject in need thereof an oxygen therapeutic composition. The oxygen therapeutic composition includes a fluorocarbon compound comprising about 4 to about 8 carbon atoms, water and saline in a concentration from about 0.6% w/v to about 1.5% w/v. The oxygen therapeutic composition is an emulsion of particles comprising the fluorocarbon and is characterized by an osmolality in the range from about 200 milliosmoles/kg to about 500 milliosmoles/kg. In certain embodiments, the fluorocarbon is a perfluorocarbon compound.

In certain embodiments, the perfluorocarbon compound is selected from the group consisting of perfluorobutane, perfluoropentane and perfluorohexane.

In certain embodiments, the oxygen therapeutic composition has an osmolality in the range from about 240 milliosmoles/kg to about 340 milliosmoles/kg.

Saline is preferably present at a concentration from about 0.6 w/v % to about 1.5 w/v %, more preferably at a concentration from about 0.7 w/v % to about 1.2 w/v %, even more preferably at a concentration from about 0.8 w/v % to about 1.0 w/v %.

In certain embodiments, the oxygen therapeutic composition is characterized by particle sizes: IWMD in the range from about 100 nm to about 600 nm (e.g., from about 100 nm to about 500 nm, from about 100 nm to about 400 nm, from about 100 nm to about 300 nm, from about 200 nm to about 600 nm, from about 300 nm to about 600 nm, from about 400 nm to about 600 nm, from about 200 nm to about 400 nm, from about 300 nm to about 500 nm).

In certain embodiments, the subject is allergic to or is at risk of suffering allergic reactions to a viscogen.

In certain embodiments, the oxygen therapeutic composition further includes one or more viscogens selected from a polyether and a polyol.

In certain embodiments, the polyether is a polyethylene glycol having an average molecular weight Mₙ less than 1,000.

In certain embodiments, the polyol is maltitol.

In yet another aspect, the invention generally relates to a method for making the oxygen therapeutic composition disclosed herein.

In certain embodiments, the oxygen therapeutic composition of the invention has from about 0.5 to about 20 w/v % of fluorocarbon. In certain embodiments, the composition has between about 1 and about 10 w/v % fluorocarbon. In certain embodiments, the composition has between about 1 and about 5 w/v % fluorocarbon. In certain embodiments, the composition has between about 5 and about 10 w/v % fluorocarbon. In certain embodiments, the composition has less than about 5 w/v % fluorocarbon. In certain embodiments, the composition has between about 1 and about 3 w/v % fluorocarbon. In certain embodiments, the composition has between about 2 and about 4 w/v % fluorocarbon. In certain embodiments, the composition has between about 3 and about 5 w/v % fluorocarbon.

In certain embodiments, the oxygen therapeutic composition comprises one or more phospholipids having carbon chains ranging from about 12 carbons to about 18 carbons in length (e.g., 12, 13, 14, 15, 16, 17 or 18 carbons in length).

In certain embodiments, the phospholipids account for a weight percent in the pharmaceutical composition from about 0.10 w/v % to about 7.5 w/v %.

Any suitable therapeutically effective dosage may be employed, for example, a dosage that ranges from about 2.0% to about 4.0%. In certain embodiments, the therapeutically effective dosage ranges from about 4.0% to about 6.0%.

In certain embodiments, a dose of about 0.5 mg/Kg to about 5 mg/Kg is administered. In certain embodiments, a dose of about 1.0 mg/Kg to about 3.5 mg/Kg is administered. In certain embodiments, a dose of about 1.5 mg/Kg to about 2.5 mg/Kg is administered. In certain embodiments, a dose of about 2.0 mg/Kg is administered.

In certain embodiments, a dose is repeated from about 60 min. to about 120 min. (e.g., about 60 min. to about 90 min., about 90 min. to about 120 min., about 60 min., about 90 min., about 120 min.) apart for 2, 3, 4, 5 or 6 times. In certain embodiments, the dose is repeated from about 90 min. to about 120 min. apart for 2 times. In certain embodiments, the dose is repeated from about 90 min. to about 120 min. apart for 3 times. In certain embodiments, the dose is repeated from about 90 min. to about 120 min. apart for 4 times. In certain embodiments, the dose is repeated from about 90 min. to about 120 min. apart for 5 times. In certain embodiments, the dose is repeated from about 90 min. to about 120 min. apart for 6 times.

Any suitable therapeutically effective dosage unit dosage form may be employed, for example, comprising about 2% to about 4% of the fluorocarbon. In certain embodiments, the unit dosage form comprises about 4% to about 6% of the fluorocarbon. In certain embodiments, the unit dosage form comprises from about 7 mg to about 150 mg (e.g., about 7 mg to about 100 mg, about 7 mg to about 70 mg, about 7 mg to about 35 mg, about 35 mg to about 150 mg, about 70 mg to about 150 mg, about 35 mg to about 70 mg) of fluorocarbon.

In certain embodiments, the oxygen therapeutic composition is administered IV to treat sickle cell disease, or a related disease or condition. In certain embodiments, the dose is between about 1 mg/Kg to about 20 mg/Kg (e.g., about 1 mg/Kg to about 15 mg/Kg, about 1 mg/Kg to about 10 mg/Kg, about 1 mg/Kg to about 5 mg/Kg) fluorocarbon.

In the case of the 2% w/vol DDFP emulsion, the dose is from about 0.01 mL/Kg to about 1.0 mL/Kg. In certain embodiments, the dose is from about 0.05 mL/Kg to about 0.5 mL/Kg fluorocarbon to treat a human patient. In certain embodiments, the dose is from about 0.05 mL/Kg to about 0.1 mL/Kg fluorocarbon to treat a human patient. In certain embodiments, the dose is from about 0.01 mL/Kg to about 0.3 mL/Kg fluorocarbon to treat a human patient. In certain embodiments, the dose is from about 0.1 mL/Kg to about 0.5 mL/Kg fluorocarbon to treat a human patient.

In certain embodiments, the oxygen therapeutic composition may be administered as an IV bolus. In certain embodiments, the oxygen therapeutic composition may be administered by sustained IV infusion. The concentration of fluorocarbon in the oxygen therapeutic composition can be increased, for example, up to about 60% w/vol if desired, to minimize the volume injected.

Hemolysis is a common condition present in SCC. The oxidized byproduct of hemolysis, hemin, exacerbates the symptoms associated with SCC in animal models in a process involving TLR4 signaling. In certain embodiments, the oxygen therapeutic composition may be co-administered with anti-inflammatory agents to ameliorate the sequelae of sickle crisis.

The oxygen therapeutic composition of the invention may be co-administered with one or more other suitable agents, or one or more such other agents may be incorporated into the fluorocarbon nanoemulsion. For example, a TLR4 inhibitor may be co-administered or incorporated into the fluorocarbon nanoemulsion of the invention. Examples of such agents include TAK-242 (with the trade name Resatorvid), a small-molecule-specific inhibitor of Toll-like receptor (TLR) 4 signaling, which has been shown to inhibit the production of NO and pro-inflammatory cytokines. TAK-242 acts by blocking the signaling mediated by the intracellular domain of TLR4, but not the extracellular domain. TAK-242 potently suppresses both ligand-dependent and-independent signaling of TLR4.

Another example of a TLR4 inhibitor is C34 (a.k.a. TLR4-IN-C34, with the formula C₁₇H₂₇NO₉), which can be used co-administered or incorporated with the fluorocarbon of the invention. Other TRL4 inhibitors that may be used in the invention include amitriptyline, cyclobenzaprine, ibudilast, imipramine, ketotifen, mianserin, naloxone, naltrexone, (+)-naltrexone, propentofylline, LPS-RS and (+)-naloxone.

OxPAPC inhibits TLR2 and LR4. It is generated by the oxidation of 1-palmitoyl-2-arachidonyl-sn-glycero-3-phosphorylcholine (PAPC), which results in a mixture of oxidized phospholipids containing either fragmented or full length oxygenated sn-2 residues. OxPAPC has been shown to inhibit the signaling induced by bacterial lipopeptide and lipopolysaccharide (LPS). OxPAPC acts by competing with CD14, LBP and MD2, the accessory proteins that interact with bacterial lipids, thus blocking the signaling of TLR2 and TLR4. PAPC, 1-palmitoyl-2-arachidonyl-sn-glycero-3-phosphorylcholine, can be incorporated into the nanoemulsion stabilizing the fluorocarbon. In certain embodiments, OxPAPC can be co-administered with the FC to improve treatment of SCC.

Hemopexin, also known as the beta-1B-glycoprotein, is a protein that scavenges and binds heme more tightly than any other protein. In certain embodiments, Hemopexin may be co-administered with the fluorocarbon nanoemulsion of the invention to improve treatment of SCC.

Antioxidants may also be used in the invention to improve the activity of the fluorocarbon. Examples of useful antioxidants include n-acetylcysteine, ascorbic acid, and α-tocopherol. In certain embodiments, n-acetylcysteine can be administered at 150 mg/Kg for 30 min then 20 mg/Kg/h plus bolus doses of 1 g ascorbic acid and 400 mg α-tocopherol.

### Examples

### Fluorocarbon Emulsions Using Different Polyethylene Glycols (PEGs) as the Viscogen

Experiments were performed to characterize formulations wherein different PEGs were employed as the viscogen. Molarity of the PEG of interest (PEG400-0.259M, PEG1000-0.166M, PEG2000-0.100M and PEG4000-0.0524M) to produce a solution with an osmotic pressure of 0.77 MPa (˜300 milliosmolar, iso-osmotic) was calculated using the equations given by N. Money (Money, N. 1989 Plant Physiol. 91, 766-769.). The contribution of DDFP and the surfactant to the osmolality or osmolarity of the solution is negligible.

The combined ingredients in 5 mL vials were prepared by adding water, PEG-Telomer B (PTB) and DDFP to one vial and sealing and crimping the cap. Then, an aqueous PEG solution (volume 3.75 mL) at double the required concentration of PEG to achieve an osmolarity of about 300 milliosmoles/kg was prepared. The solution of the PEG was then added to the vial containing DDFP, PTB and water using a 5/6 mL Henke Sass Wolf NormJect® syringe equipped with a 25 gauge needle. The final concentration of components in the vial was DDFP 2% wt/vol, PTB 0.3% wt/vol, and the respective PEG concentrations.

The vials were vortexed 2×30 sec (1× upright, 1× inverted) at speed 9.5 on a mini-vortexer. The vials were allowed to stand 2 min and then sonicated in a VWR Aquasonic 75HT ultrasonic cleaning bath with vial located in the center position with respect to the front, rear and sides of the ultrasonic bath. It is noted that in certain experiments on sucrose-containing DDFPe formulations it was found that vortexing for 3×3 sec upright and 3×3 sec inverted dispersed the insoluble components in the aqueous bulk phase sufficiently such that post sonication the material was converted to nanodroplets efficiently. This served to minimize foam due to vortexing. The concern about foam generated by vortexing was that the foam might entrain a significant fraction of DDFP hence limiting its availability to form surfactant-coated nanodroplets. The change in protocol was effective in that vortexing for 2×30 sec produces a large amount of foam that is dissipated little during the sonication period. Sonication time was generally 8 min, but exceptions are noted in notes on each sample.

Particle sizing was performed by addition of the material to a cuvette containing 3 mL of 0.2 micron-filtered 0.9% NaCl at 20° C. followed by gentle inversion 10×, insertion of the cuvette into the instrument and temperature equilibrating the sample for 8 min. For comparison, a DDFP emulsion employing sucrose is shown in Table 1.

TABLE 1
DDFP emulsion (NOX-108) formulation

| | | | Concentration |
|---|---|---|---|
| Ingredient | Specification | Purpose | (mg/mL) |
| Dodecafluoropentane | Medical Grade | Active | 20 |
| Sucrose | Medical Grade | Excipient | 300 |
| PEG-Telomer B | Purified Chemical | Excipient | 3 |
| | Grade | | |
| Water for Injection | USP | Solvent | q.s. to 1 mL |
| Nitrogen | Medical Grade | Head space | q.s. |
| | | Air Flush | |
| Sodium Phosphate | USP | Buffer | 0.01M |
| Hydrochloric acid | USP | Excipient | q.s. |

In experiments with PEG4000-and PEG2000-based formulations, it was found that post sonication the material settles very quickly, resulting in a heavy sludge-like material that gives very low scattering intensity, large intensity weighted mean diameter (IWMD) and volume-weighted mean diameter (VWMD) yet very low number weighted mean diameter, as well as very wide distributions with high chi-squared values.

In experiments with PEG1000-based formulations, it was found that they settle less quickly than the PEG2000-and PEG4000-based formulations. Sonication gave acceptable particle size parameters and the smallest IWMD at ˜132 nm (vs ˜200 nm for PEG400 based particles). The particle size obtained at 8 min sonication and 10.5 min sonication for PEG1000-based formulation were extremely close, suggesting that 8 min may already be the saturation value for sonication of that formulation.

For the PEG400-based formulation, the particle size values at 8 min and 10 min were very close suggesting that 8 min may be either the saturation sonication time or very close to it. In fact, the 8 min IWMD was slightly smaller than that obtained at the 10 min sonication time. This variation may be due to slight differences in positioning of the vial in the bath. The PEG400-based formulation provides the most scattering intensity per unit sample.

These results show suitable PEGs as osmolytes for formulations include PEG400 and PEG1000, with PEG400 being preferred. It is also noted that, based on the results herein, PEG200 and PEG300 are suitable PEGs in these formulations as well.

TABLE 2
Isotonic Formulation Made Using PEG400-Particle Size Values

| | | | | | IW | | | | |
|---|---|---|---|---|---|---|---|---|---|
| | | | | | 99% | | VW99% | | |
| Sample/Procedure | | IWMD | IWSD | % SD | Dist < | VWMD | Dist < | NWMD | |
| Details | Vial | (nm) | (nm) | IWMD | (nm) | (nm) | (nm) | (nm) | Xi² |
| PEG400 viscogen, | 10 | 201.8 | 65.6 | 32.5 | 407.5 | 173.5 | 350.30 | 114.30 | 0.27 |
| osmolyte; 10 min | | | | | | | | | |
| sonication | | | | | | | | | |
| PEG400 viscogen, | 11 | 192.4 | 64.8 | 33.7 | 397.8 | 159.3 | 329.50 | 103.00 | 0.51 |
| osmolyte; 8 min | | | | | | | | | |
| sonication | | | | | | | | | |

| | | | | | | | | |
|---|---|---|---|---|---|---|---|---|
| Average | 197.1 | 65.2 | 33.1 | 402.7 | 166.4 | 339.9 | 108.7 | 0.39 |
| SD | 6.65 | 0.57 | 0.85 | 6.9 | 10.0 | 14.7 | 8.0 | 0.17 |

TABLE 3
Isotonic Formulations Made Using PEG1000-Particle Size Values

| | | | | | | IW | | VW99% | | |
|---|---|---|---|---|---|---|---|---|---|---|
| Sample | | Measure | | | | 99% | | CUM | | |
| Procedure- | | time | IWMD | IWSD | % SD | Dist < | VWMD | Dist < | NWMD | |
| Details | Vial | (min) | (nm) | (nm) | IWMD | (nm) | (nm) | (nm) | (nm) | Xi² |
| PEG1000 | 8 | 15 | 134.1 | 45.1 | 33.6 | 276.9 | 101.1 | 208.7 | 69.8 | 0.27 |
| viscogen, | | | | | | | | | | |
| osmolyte; 8 | | | | | | | | | | |
| min | | | | | | | | | | |
| sonication | | | | | | | | | | |
| PEG1000 | 9 | 15 | 132.4 | 43.0 | 32.5 | 267.4 | 101.6 | 205.1 | 71.9 | 0.40 |
| viscogen, | | | | | | | | | | |
| osmolyte; 10.5 | | | | | | | | | | |
| min | | | | | | | | | | |
| sonication | | | | | | | | | | |

| | | | | | | | | |
|---|---|---|---|---|---|---|---|---|
| Average | 133.3 | 44.1 | 33.0 | 272.2 | 101.4 | 206.90 | 70.9 | 0.34 |
| SD | 1.20 | 1.48 | 0.78 | 6.72 | 0.35 | 2.55 | 1.48 | 0.09 |

TABLE 4
Isotonic Formulation of DDFPe Using PEG2000-Particle Size Values

| Sample | | | | | | | VW99% | | |
|---|---|---|---|---|---|---|---|---|---|
| Procedure- | | IWMD | IWSD | % SD | IW99% | VWMD | CUM | NWMD | |
| Details | Vial | (nm) | (nm) | IWMD | Dist< | (nm) | Dist < (nm) | (nm) | Xi² |
| PEG2000 | 6 | 256.1 | 171.1 | 66.8 | 967.2 | 139.7 | 531.4 | 37.6 | 1.00 |
| viscogen, | | | | | | | | | |
| osmolyte; 8 | | | | | | | | | |
| min | | | | | | | | | |
| sonication | | | | | | | | | |
| PEG4000 | 4 | 512.8 | 431.3 | 84.1 | 2497.0 | 673.8 | 3137.8 | 67.0 | 4.72 |
| viscogen, | | | | | | | | | |
| osmolyte; 8 | | | | | | | | | |
| min | | | | | | | | | |
| sonication | | | | | | | | | |

### Formulation of the Fluorocarbon Emulsion Using 10% w/v Sucrose as the Viscogen and Osmolyte

Studies using 10% sucrose as the continuous phase were also performed.

Experiments were performed to characterize formulation variants where the sucrose viscogen concentration is reduced to 10% wt/vol vs 30% wt vol.

The combined ingredients in 5 mL vials were prepared by adding water, PEG-Telomer B (PTB) and DDFP to one vial and sealing and crimping the cap. Then, an aqueous solution of 20% wt/vol sucrose (double the required concentration of sucrose required) was added to separate 5 mL vials (volume 3.75 mL). The solution of the sucrose was then added to the vial containing DDFP, PTB and water using a 5/6 mL Henke-Sass Wolf NormJect® syringe equipped with a 25 gauge needle. The final concentration of components in the vial was DDFP 2% wt/vol, PTB 0.3% wt/vol, sucrose 10% w/v.

The first vial was vortexed 2×30 sec (1× upright, 1× inverted) at speed 9.5 on a mini-vortexer. Vial 24 was vortexed 10 sec upright and 10 sec inverted whereas Vial 12 was vortexed 3×3 sec upright and 3×3 sec inverted prior to sonication. The vials were then allowed to stand 2 min and then sonicated in a VWR Aquasonic 75HT in the center position with respect to the front, rear and sides of the ultrasonic bath. Note that vortexing for 3×3 sec upright and 3×3 sec inverted dispersed the insoluble components in the aqueous bulk phase sufficiently such that post sonication the material was converted to nanodroplets efficiently. This serves to minimize foam due to vortexing.

Particle sizing was carried by addition of the material to a cuvette containing 3 mL of 0.2 micron filtered 0.9% NaCl at 20° C. and gentle inversion 10×, insertion of the cuvette into the instrument equilibrating for 8 min. Then the neutral density filter was initialized followed by initiation of the measurement using autoset of the ND filter to 250 KHz intensity.

The concern that foam generated by vortexing may entrain a significant fraction of DDFP hence limiting its availability to form surfactant-coated nanodroplets was behind experimenting with shorter vortexing times. The change in protocol was effective in that vortexing for 2×30 sec produces a large amount of foam which is dissipated little during the sonication period whereas the shorter vortexing regimes did not and also provided sonicated material with the expected properties. Sonication time was 8 min for each vial. Sonication gave acceptable particle size parameters and the IWMD was 209.5 +/−10.2 nm, VWMD was 181.8 nm +/−8.6 nm, NWMD was 116.4 nm +/−11.4 nm, SD % was 33.1% +/−3.6% and Xi² was 0.27+/−0.09. These are typical parameters found by sonication of vials when 30% sucrose is the continuous phase.

The results showed that a 10% w/v sucrose-based formulation can be prepared and gives acceptable particle size values for DDFPe oxygen delivery. Data from samples on the day of preparation are presented below.

TABLE 5
Isotonic Formulation of DDFP emulsion using 10% Sucrose-Particle Size Values

| Sample/ | | Measure | | | | IW 99% | | VW99% | | |
|---|---|---|---|---|---|---|---|---|---|---|
| Procedure | | time | IWMD | IWSD | % SD | Dist < | VWMD | Dist < | NWMD | |
| Details | Vial | (min) | (nm) | (nm) | IWMD | (nm) | (nm) | (nm) | (nm) | Xi² |
| Vortex 30 s | 22 | 15 | 221.2 | 80.5 | 36.4 | 482.5 | 191.1 | 416.8 | 108.3 | 0.27 |
| upright and | | | | | | | | | | |
| inverted, speed | | | | | | | | | | |
| 10 | | | | | | | | | | |
| Vortex 10 sec | 24 | 15 | 202.7 | 59.4 | 29.3 | 383.7 | 180.1 | 383.7 | 129.4 | 0.18 |
| upright, 10 sec | | | | | | | | | | |
| inverted speed | | | | | | | | | | |
| 5.5; | | | | | | | | | | |
| Vortex 3 × 3 | 12 | 15 | 204.5 | 68.7 | 33.6 | 422.1 | 174.1 | 361.0 | 111.4 | 0.35 |
| sec upright, | | | | | | | | | | |
| 3 × 3 sec | | | | | | | | | | |
| inverted speed | | | | | | | | | | |
| 5.5 | | | | | | | | | | |

| | | | | | | | | |
|---|---|---|---|---|---|---|---|---|
| Average | 209.5 | 69.5 | 33.1 | 429.4 | 181.8 | 387.2 | 116.4 | 0.27 |
| SD | 10.2 | 10.6 | 3.6 | 49.8 | 8.6 | 28.1 | 11.4 | 0.09 |
| RSD % | 4.9 | 15.2 | 10.8 | 11.6 | 4.7 | 7.3 | 9.8 | 31.9 |

The isotonic emulsion has good stability characteristics. The already-sampled vials (above) were allowed to stand for 9 days post sampling and then resampled on Day 9. Data for these measurements appear in Table 6 and are well within the acceptable range of particle size.

The incremental change in particle size parameters from Day 1 to Day 9 is shown in Table 7. It is noted that these vials stood 9 days with punctured stoppers and an increased headspace, a condition under which particle growth is faster than when vials with intact (not punctured) stoppers stand for the same period.

TABLE 6
Isotonic Formulation of DDFPe Resampled on Day 9

| Sample | | Measure | | | | IW 99% | | | | |
|---|---|---|---|---|---|---|---|---|---|---|
| Procedure- | | time | IWMD | IWSD | % SD | Dist < | VWMD | VW99% | NWMD | |
| Details | Vial | (min) | (nm) | (nm) | IWMD | (nm) | (nm) | Dist < (nm) | (nm) | Xi² |
| Vortex 30s | 22 | 15 | 286.3 | 124.2 | 43.4 | 714.6 | 278.7 | 695.7 | 106.4 | 0.30 |
| upright and | | | | | | | | | | |
| inverted, | | | | | | | | | | |
| speed 10 | | | | | | | | | | |
| Vortex 10 | 24 | 15 | 324.7 | 157.8 | 48.6 | 893.2 | 334.7 | 921.1 | 93.5 | 0.52 |
| sec upright, | | | | | | | | | | |
| 10 sec | | | | | | | | | | |
| inverted | | | | | | | | | | |
| speed 5.5 | | | | | | | | | | |
| Vortex 3 × 3 | 12 | 15 | 283.0 | 131.9 | 46.6 | 750.3 | 265.1 | 702.7 | 86.8 | 0.27 |
| sec upright, | | | | | | | | | | |
| 3 × 3 sec | | | | | | | | | | |
| inverted | | | | | | | | | | |
| speed 5.5 | | | | | | | | | | |

| | | | | | | | | |
|---|---|---|---|---|---|---|---|---|
| Average | 298.0 | 138.0 | 46.20 | 786.0 | 292.8 | 773.2 | 95.6 | 0.36 |
| SD | 23.2 | 17.6 | 2.6 | 94.5 | 36.9 | 128.2 | 10.0 | 0.14 |
| RSD % | 7.8 | 12.8 | 5.7 | 12.0 | 12.6 | 16.6 | 10.4 | 37.6 |

TABLE 7
% Change in Particle Size Parameters over 9-Day Period for Previously Sampled
Vials of DDFPe Formulation Prepared using 10% Sucrose as the Viscogen and Osmolyte

| | IWMD | IWSD | % SD | IW 99% of | VWMD | VW99% of | NWMD | |
|---|---|---|---|---|---|---|---|---|
| Parameter | (nm) | (nm) | IWMD | Dist < (nm) | (nm) | Dist < (nm) | (nm) | Xi² |
| % Change | 42.3 | 98.4 | 39.6 | 83.0 | 61.1 | 99.7 | −17.9 | 36.25 |
| Day 1-Day 9 | | | | | | | | |

### Formulation of the Fluorocarbon Emulsion Using 10% w/v Maltitol as the Viscogen and Osmolyte

When maltitol at the level of 10% w/v is employed as the osmolyte and viscogen in the formulation of the emulsion, an isotonic or nearly isotonic solution is produced. It may be advantageous to employ maltitol instead of sucrose especially in the case of diabetic patients, as the glyceimic index and the insulinemic index are about half of that of sucrose (Livesey, G. “Health potential of polyols as sugar replacers, with emphasis on low glycaemic properties”. Nutrition Research Reviews 2003, 16, 163-191).

Wheaton nominal 5 mL capacity serum (total capacity 9 mL) vials with 20 mm opening, were charged with a 0.383 mL aliquot of a solution of PEG Telomer B surfactant (6% w/v) in deionized UV light sterilized 18 MΩ water (solution A), followed by a solution of 10 mM NaH₂PO₄/Na₂HPO₄ buffer (pH 7.1) containing Maltitol (98% purity, Sigma Aldrich Co. Cat No M8892) 10% w/v (solution B). Solution B was filtered into the vials through a Pall Sciences 0.2 micron GH Polypro Syringe filter. The vials were stoppered with Wheaton chlorobutyl 20 mm stoppers. Then the vials were fitted with crimp caps and weighed on an analytical balance to the nearest 0.1 milligram. Following this, each vial was charged with a 0.153 g aliquot of dodecafluoropentane (DDFP) and the caps were manually crimped immediately after dispensing the aliquot of DDFP into each vial. The vials were weighed after dispensing of the DDFP and crimp capping.

TABLE 8
Amount and Accuracy of Dispensing of
DDFP into Vials Containing Maltitol (10% w/v),
Peg Telomer B Fluorosurfactant and Phosphate Buffer

| | Vial ID | | |
|---|---|---|---|
| | VM-1 | VM-2 | VM-3 |
| Weight Vial, solutions A and B, | 18.3145 | 18.3603 | 18.2710 |
| stopper and crimp cap (g) | | | |
| Weight Vial, solutions A and B, | 18.4724 | 18.5252 | 18.4253 |
| stopper, crimp cap and aliquot of | | | |
| DDFP (g) | | | |
| Weight added DDFP (mg) | 157.0 | 164.9 | 154.3 |
| % Theory of DDFP | 103.2 | 107.8 | 100.8 |

Prior to sonication each vial was vortexed for 30 sec upright and 30 sec inverted. Each vial was sonicated for 8 min using a VWR Aquasonic 75HT sonication bath at a frequency of 40 KHz with the vial in the center position with respect to the front, rear and sides of the ultrasonic bath and immersed such that the liquid layer of the vial and that of the bath were coincident. The vials were allowed to stand for 20 h. Then the particle size distribution was determined using a Brookhaven Instruments NanoBrook 90Plus dynamic light scattering particle sizer. Values for the intensity weighted, volume-weighted and number weighted size distributions are given in Table 9.

TABLE 9
Isotonic Formulation of DDFPe using 10% Maltitol as Viscogen and
Osmolyte-Particle Size Values

| | Measure | | | | IW 99% | | |
|---|---|---|---|---|---|---|---|
| | time | IWMD | IWSD | % SD | Dist < | VWMD | NWMD |
| Vial ID | (min) | (nm) | (nm) | IWMD | (nm) | (nm) | (nm) |
| VM-1 | 20 | 251.91 | 85.8 | 34.06 | 515.37 | 191.46 | 137.75 |
| VM-2 | 20 | 249.71 | 84.7 | 33.91 | 50948 | 190.21 | 137.22 |
| VM-3 | 20 | 253.61 | 79.39 | 31.30 | 492.93 | 200.75 | 151.66 |
| Average | | 251.74 | 83.29 | 33.09 | 505.93 | 194.14 | 142.21 |
| SD | | 1.96 | 3.42 | 1.55 | 11.64 | 5.76 | 8.18 |
| RSD | | 0.78 | 4.11 | 4.68 | 2.30 | 2.97 | 5.76 |
| (%) | | | | | | | |

The data for emulsions produced by sonication using maltitol as the viscogen are well within the acceptable range and very similar to those obtained using 10% sucrose as the viscogen and osmolyte, the higher glycemic index and insulinemic index sucrose can be replaced with maltitol as osmolyte and viscogen.

Other sugar or sugar alcohol compounds can be employed as for maltitol. For example, erythritol, xylitol, sorbitol, mannitol, isomalt, lactitol and polyglycitol can be employed singly or in combination in order to tune the viscosity of the formulation while maintaining an isotonic formulation and a low overall glycemic index and insulinemic index for the formulation.

### Preparation of the Fluorocarbon Emulsion Using 0.9% Saline as the Osmolyte

An aqueous solution of PEG Telomer B fluorosurfactant (5%) in sterile water for injection was stirred in a glass vessel cooled to 2-4° C. under a nitrogen atmosphere. Then DDFP was added to the solution and the mixture stirred for 1 h under a nitrogen pressure of 10 PSI at 2-4° C. Then, this mixture was homogenized using an Avestin C50 high pressure homogenizer for 18 minutes. The resulting homogenizate was dissolved in 1.0% saline solution (1.0% w/v NaCl) to give a final composition of about 0.3% w/v PEG Telomer B and about 2% DDFP. The resulting solution was aliquoted into Wheaton nominal 10 mL serum vials which were stored at ambient temperature. Particle sizing as described for samples containing 10% sucrose was conducted over a 3 months period. Data for an average of 5 vials for each time point are shown in Table 10.

TABLE 10
Isotonic Formulation of DDFPe using 0.9% w/v
Saline as Osmolyte - Particle Size Values

| | Intensity | | | Intensity |
|---|---|---|---|---|
| | Weighted | Standard | | Weighted 99% |
| | Mean | Deviation | % Standard | Cumulative |
| | Diameter | of IWMD | Deviation of | Distribution |
| Time point | (IWMD) nm | (nm) | IWMD | (nm) |
| Release (Day | 285 | 91 | 31.9 | 569 |
| 1) | | | | |
| 3 Weeks | 282 | 135 | 47.9 | 756 |
| 3 Months | 283 | 129 | 45.5 | 736 |

The data of table 10 demonstrate the stability of an isotonic formulation of the perfluorocarbon emulsion and that, where needed, the emulsion can be prepared and remain stable in the absence of a viscogen. The stability of the particles in absence of a viscogen is an unexpected finding; viscogens are employed to stabilize emulsions with respect to sedimentation and to particle size growth. Such a formulation can be beneficial for administration to diabetic patients and those who may be allergic or have adverse reactions to any given viscogen.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### Incorporation by Reference

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### Equivalents

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1-65. (canceled)

66. An oxygen therapeutic composition, comprising:
a fluorocarbon compound selected from the group consisting of perfluorobutane, perfluoropentane and perfluorohexane;
PEG-Telomer B (PTB) as a surfactant;
a viscogen at a concentration between about 2 w/v % to about 15 w/v % selected from
polyethylene glycol (PEG) with average molecular weight Mₙ between about 200 and about 400, and
maltitol; and
water,
wherein the oxygen therapeutic composition
is an emulsion of particles comprising the fluorocarbon and having particle sizes: intensity weighted mean diameter (IWMD) in the range from about 100 nm to about 600 nm, and
is characterized by an osmolality in the range from about 275 milliosmoles/kg to about 305 milliosmoles/kg.

67. The oxygen therapeutic composition of claim 66, wherein the viscogen is a polyethylene glycol (PEG).

68. The oxygen therapeutic composition of claim 66, wherein the viscogen is maltitol.

69. The oxygen therapeutic composition of claim 66, wherein the composition is free of sucrose.

70. The oxygen therapeutic composition of claim 66, wherein the composition comprises sucrose.

71. The oxygen therapeutic composition of claim 70, wherein sucrose is present at a concentration less than about 15 w/v %.

72. The oxygen therapeutic composition of claim 66, wherein the particles have particle sizes: IWMD in the range from about 200 nm to about 500 nm.

73. An oxygen therapeutic composition, comprising:
a fluorocarbon compound selected from the group consisting of perfluorobutane, perfluoropentane and perfluorohexan;
PEG-Telomer B (PTB) as a surfactant; and
NaCl in a concentration from about 0.7% w/v to about 1.2% w/v, wherein the oxygen therapeutic composition
is an emulsion of particles comprising the fluorocarbon and having particle sizes: intensity weighted mean diameter (IWMD) in the range from about 100 nm to about 600 nm, and
is characterized by an osmolality in the range from about 275 milliosmoles/kg to about 305 milliosmoles/kg,
wherein the composition is free of a viscogen and is free of sucrose.

74. The oxygen therapeutic composition of claim 73, further comprising maltitol.

75. A method for treating sickle cell disease, or a related disease or condition, comprising administering to a subject in need thereof an oxygen therapeutic composition comprising: a fluorocarbon compound comprising about 4 to about 8 carbon atoms, a surfactant, a viscogen and water, wherein the oxygen therapeutic composition is characterized by an osmolality in the range from about 200 milliosmoles/kg to about 500 milliosmoles/kg.

76. A method for treating sickle cell disease, or a related disease or condition, comprising administering to a subject in need thereof an oxygen therapeutic composition comprising:
a fluorocarbon compound comprising about 4 to about 8 carbon atoms;
a surfactant; and
saline in a concentration from about 0.6% w/v to about 1.5% w/v, wherein the oxygen therapeutic composition
is an emulsion of particles comprising the fluorocarbon, and
is characterized by an osmolality in the range from about 200 milliosmoles/kg to about 500 milliosmoles/kg.
